(19) United States
(12) Patent Application Publication
(10) Pub. No.: US 2017/0039480 A1
(43) Pub. Date: Feb. 9, 2017

(54) Workout Pattern Detection

(71) Applicant: Microsoft Technology Licensing, LLC, Redmond, WA (US)

(72) Inventors: Hadas Bitran, Ramat Hasharon (IL); Elinor Axelrod, Hod Hasharon (IL); Gal Lavee, Tel Aviv (IL)

(21) Appl. No.: 15/007,938

(22) Filed: Jan. 27, 2016

Related U.S. Application Data
(60) Provisional application No. 62/202,111, filed on Aug. 6, 2015.

(51) Int. Cl. G06N 7/00; G06N 5/04; G06N 99/00; A61B 5/024; A61B 5/01; A61B 5/11; G06F 17/30; A63B 24/00

(52) U.S. Cl. CPC G06N 7/005; G06F 17/30312; G06N 5/048; A63B 24/0062; A63B 24/0075; A61B 5/02438; A61B 5/01; A61B 5/11; G06N 99/005

(57) ABSTRACT

Aspects of the technology described herein can analyze signal data from multiple computing devices to ascertain a user's exercise pattern. Understanding a user's exercise pattern can help reduce power usage by automatically turning physiological sensors on and off to coincide with the start and end of an exercise event. Exemplary computing devices that can provide signal data related to a user's exercise routine can include a mobile computing device (e.g., smart phone) that captures location signals and other contextual data and a wearable computing device (e.g., fitness tracker) that captures physiological characteristics of the user, such as heart rate, temperature, and movement. The signals captured by the multiple computing devices can be analyzed together to determine when an individual exercise event has occurred. The plurality of exercise events associated with a user or group of users can be analyzed to ascertain an exercise pattern.

### CROSS-REFERENCE TO RELATED APPLICATIONS

This application claims the benefit of U.S. Provisional Application No. 62/202,111, titled “Workout Pattern Detection,” filed Aug. 6, 2015, which is hereby expressly incorporated by reference in its entirety.

### BACKGROUND

People often wish to track their exercise routines using computing devices. Computing devices can also generate numerous experiences related to workouts, including reminders, progress tracking, planning, etc. Any single computing device may not have access to all available signals that can determine when a workout occurred. Some workout signals can include human error that causes the signal to be an inaccurate representation of an actual workout. Computing devices that rely on incomplete or inaccurate signals can fail to generate accurate experiences for the user.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In various aspects, systems, methods, and computer-readable storage media are provided to improve a computing device's ability to accurately extract contextual features from signal data. Aspects of the technology described herein can analyze signal data from multiple computing devices to ascertain a user's exercise pattern. Understanding a user's exercise pattern can help reduce power usage by automatically turning components, such as physiological sensors, on and off to coincide with the start and end of an exercise event (including a predicted, future exercise event). Exemplary computing devices that can provide signal data related to a user's exercise routine can include a mobile computing device (e.g., smart phone) that captures location signals and other contextual data and a wearable computing device (e.g., fitness tracker) that captures physiological characteristics of the user, such as heart rate, temperature, and movement. The signals captured by the multiple computing devices can be analyzed together to determine when an individual exercise event has occurred. The plurality of exercise events associated with a user or group of users can be analyzed to ascertain an exercise pattern. The exercise pattern and associated context can then be used to generate exercise-related experiences for the user.

### BRIEF DESCRIPTION OF THE DRAWINGS

The technology described herein is illustrated by way of example and not limitation in the accompanying figures in which like reference numerals indicate similar elements and in which:

FIG. 1 is a block diagram of an example operating environment suitable for implementations of the present disclosure;

FIG. 2 is a diagram depicting an example computing architecture suitable for implementing aspects of the present disclosure;

FIGS. 3-5 are flow diagrams showing additional exemplary methods of inferring an exercise pattern, in accordance with an aspect of the technology described herein;

FIG. 6 is a block diagram of an exemplary computing environment suitable for use in implementing aspects of the technology described herein; and

FIGS. 7A, 7B, and 7C depict various examples of notifications that may be presented to a user, in accordance with some embodiments of the invention.

### DETAILED DESCRIPTION

The various technology described herein are set forth with sufficient specificity to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

Aspects of the technology described herein analyze signal data from multiple computing devices to ascertain a user's exercise pattern. Exemplary computing devices that can provide signal data related to a user's exercise routine can include a mobile computing device (e.g., smart phone) that captures location signals and a wearable computing device (e.g., fitness tracker) that captures physiological characteristics of the user, such as heart rate, temperature, and movement. The signals captured by the multiple computing devices can be analyzed to determine when an individual exercise event has occurred. The plurality of exercise events associated with a user or group of users can be analyzed to ascertain an exercise pattern. The exercise pattern and associated context can then be used to generate exercise-related experiences for the user.

Each identified exercise event can be associated with contextual information directly related to the exercise event, as well as peripheral contextual information describing other activities in a user's day that are not directly related to the exercise event. The direct contextual information associated with an exercise event can comprise a location where the exercise event occurred, duration of the exercise event, physiological characteristics associated with the exercise event, the presence of other people during the exercise event, and such. The direct contextual information can be learned through analysis of signal data captured during the exercise event. The peripheral contextual information can include a description of a user's activities during the day, days, our hours before or after an exercise event. Exemplarily peripheral contextual information includes an amount of sleep the user receives prior to an exercise event, calendared events or meetings, and a user's location prior to an exercise event, including at different times of the day leading up to an exercise event.

Aspects of the technology described herein can also use semantic data describing the user. Semantic information can include a user's social contacts, work contacts, interests, home location, work address, calendar data, tasks, and other information. The semantic data can be used to identify or define an exercise event or exercise pattern, and/or generate exercise-related experiences for the user.

The technology described herein can analyze signal data to determine whether an exercise event has occurred. As mentioned, the signals can be derived from two or more computing devices. As an initial step, the signal data is analyzed to determine that an exercise event has occurred. In one aspect, the occurrence or non-occurrence of an exercise event is determined using a machine learning mechanism, such as a neural network. In addition to determining whether an exercise event occurred, the machine learning mechanism can classify the exercise event into a specific fitness exercise, such as running, walking, cycling, soccer, tennis, yoga, or such.

In another aspect, the occurrence of an exercise event or the non-occurrence of an event is analyzed or determined by comparing physiological data along with other sensor data, such as location data, against a plurality of heuristics that define various exercise events. Heuristics could be used to classify an exercise event into a specific fitness exercise. Other methods of ascertaining whether an exercise event has occurred are possible.

Aspects of the technology can combine signals from multiple sources to eliminate false positives that occur when only one signal is used to determine whether an exercise event occurred. For example, location or other contextual data could be used to cross check physiological data and physiological data can be used to cross check location and other contextual data. In isolation, either of these signals can produce false positives (event that would be classified as an exercise event, but that is not actually an exercise event).

For example, a user's heart rate may exceed a baseline by a threshold amount even when not exercising. For example, a user may be running to catch a bus, which could elevate the user's heart rate, but is not an exercise event. If only the physiological information were used, then running to catch the bus could be classified as an exercise event. Aspects the technology use other data that can be correlated with this physiological data to determine an exercise event did not occur. For example, location information could indicate that the user was located along a street, stopped running at a venue classified as a bus stop, and actually got on a bus or at least into another vehicle based on a change in device velocity. Additional contextual information could be evaluated to classify the event as a non-exercise event. For example, the location data associated with the mobile device could follow a known bus route, further confirming that the user was running to catch a bus. In this scenario, the physiological data indicating a possible exercise event is disambiguated using location data provided by the smart phone to conclude that an exercise event has not occurred.

In another example, location data indicating a possible exercise event is disambiguated using the physiological data to conclude that an exercise event has not occurred. For example, a user could be present at a venue associated with exercising, such as a gym. However, while at the gym, the physiological data could indicate that the user's heart rate did not exceed a threshold associated with exercising. In this situation, the user may be shopping in a store associated with a gymnasium, picking up children, or performing some other task that does not involve exercising.

However, when the signal data for multiple devices is consistent with an exercise event, then an exercise event description can be generated and stored in an exercise event data store. The exercise event description can include a context for the exercise event and a description of the exercise event. The context of the exercise event can include a location where the exercise event occurred, a duration of the exercise event, and other people present during the exercise event. The description of the exercise event can include a description of the exercises engaged in. For example, the exercise event could be classified as running, cycling, swimming, weightlifting, or some other event. The description can be generated by analyzing physiological data such as heart rate, along with other data gleaned from the fitness trackers. For example, a number of steps, velocities, and other movement exercise could indicate what exercise was being performed during the exercise event. In addition, semantic information known about the user could be used to determine what type of exercise was occurring. For example, a calendar entry associated with the time when the exercise event occurred could indicate yoga class, weightlifting with personal trainer, etc.

In one aspect, the contextual information for an exercise event also includes weather information. Weather information can be important contextual information because it can indicate when a probable exercise event may not occur in the future. For example, a plurality of exercise events may be analyzed to determine that the user runs every Monday, Wednesday, and Friday when the weather falls into a particular temperature range and precipitation is not forecast.

The plurality of exercise events are analyzed to determine or infer an exercise pattern for the user. A user may have multiple exercise patterns or only a single exercise pattern. For example, a first exercise pattern for the user may comprise running Monday, Wednesday, and Friday morning. A second exercise pattern for the same user can comprise weight training every Tuesday, Thursday, and Saturday morning at a particular gymnasium. The pattern can include contextual information that can qualify the pattern. For example, the user may run every Monday, Wednesday, and Friday morning when the user begins their day in their hometown. An analysis of user data may indicate that the user does not run when she is traveling. Instead, she may follow an alternate exercise routine when traveling, such as working out in a hotel exercise facility. Similarly, weather can comprise contextual information that can define exceptions to an inferred pattern. For example, a user may not run when it is raining or below a certain temperature. In these exceptional circumstances, an alternate exercise pattern may be followed, such as going to a gymnasium. All of this illustrates that an exercise pattern can be very granular and include different types of exercises at different times along with exceptions and alternatives. Once one or more patterns are established for the user, the patterns can be stored in an exercise pattern data store.

The exercise pattern data store can be accessible to one or more applications that generate exercise-related experiences. For example, a personal assistant application could access the exercise patterns to generate notifications, calendar entries, task list items, and other content. For example, the user may provide a reminder to head to the gym 30 minutes prior to a user's inferred or probable exercise event. The personal assistant could provide traffic information or a recommended departure time for the user.

In another aspect, an application could anticipate an exception to an established pattern based on the contextual information associated with the pattern. For example, an application could ascertain that the present weather context indicates that the user will not complete an anticipated exercise event. The assistant could alert the user to the contextual situation, such as forecasted rain, and suggest an alternative exercise.

In another aspect, the technology could provide automatic notifications to other people typically associated with a user's exercise event. For example, the contextual information could indicate that the user runs with four other people every Monday, Wednesday, and Friday. The participation of other people in an exercise event can be determined by evaluating information from multiple devices associated with different people. As patterns are established for each user, the patterns could be evaluated to determine overlaps that indicate joint participation.

A particular response to an anticipated exception in the exercise pattern can be learned by analyzing a user's actions, such as communication, when an exception occurs. For example, the user's communications could be analyzed to determine that a user will email other members of the group when he is not able to participate. In this situation, a personal assistant application or other application with access to the exercise pattern and contextual information could automatically generate a suggestion to send a communication to the other users when it appears that the user is not likely to participate in the exercise event. Alternatively or additionally, the personal assistant application could automatically provide a notification.

Another application could access the user's exercise pattern to suggest optimizations. The application can look at specific descriptions of the exercise events, as well as patterns. For example, a user may be exercising vigorously for only 15 minutes several days a week. The optimization application could suggest that the user would benefit more, cardiovascularly, from fewer but longer workouts. In addition, the peripheral contextual information associated with historical exercise events could be analyzed to determine circumstances that interfere with a user's workout. For example, the analysis of event description information could indicate that the user exercises for less time and less vigorously after sitting all day at work.

The technology described herein may use contextual signals to identify an exercise event or pattern. The technology can also associate contextual information with an exercise event or pattern.

“Contextual signals,” as utilized herein, may reflect any attribute of a user (for instance, physical characteristics), the user's historical interaction with the system (e.g., behavior, habits, and system interaction patterns), and/or the user's recent interaction with the system (with “recency” being defined in accordance with a predetermined time frame relative to a given point in time) that may affect the likelihood or probability that the user desires to engage with a particular computer application or computer program. Such contextual signals may include, by way of example only and not limitation, the location of the user of the computing device (determined utilizing, for instance, Global Positioning System (GPS) signals, Internet Protocol (IP) address, or the like), the time of day (either general (for instance, morning or afternoon) or exact (for instance, 6:00 pm)), the date (either exact or generally a particular month, season, etc.), a physical characteristic of the user (for instance, if the user is paralyzed and capable of only voice input, or the like), a task currently engaged in on the computing device by the user, a task recently engaged in on the computing device by the user (again with “recency” being defined in accordance with a predetermined time frame relative to a given point in time), an object the user is currently engaged with on the computing device (for instance, an entity such as a contact, a file, an image, or the like), an object the user was recently engaged with on the computing device, a function currently being performed by the user on the computing device, a function recently performed by the user on the computing device, hardware currently being utilized on the computing device, hardware recently utilized on the computing device, software currently being utilized on the computing device, and software recently utilized on the computing device.

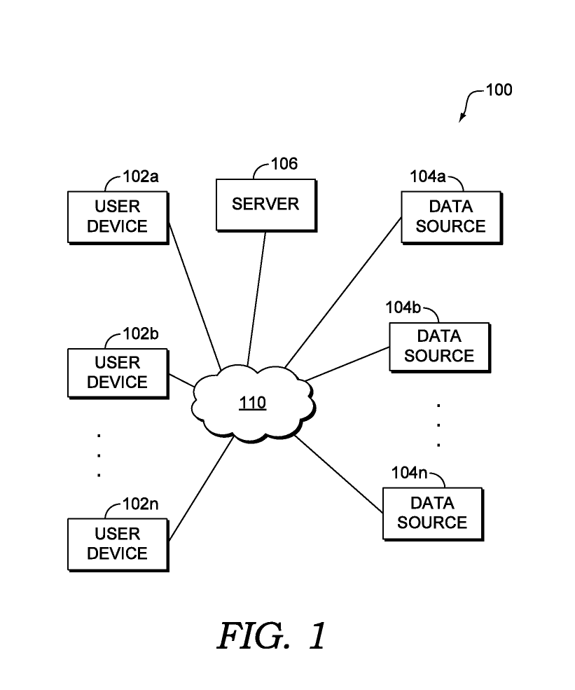

Having briefly described an overview of aspects of the technology described herein, an exemplary operating environment in which aspects of the technology described herein may be implemented is described below in order to provide a general context for various aspects. Referring to the figures in general and initially to FIG. 1 in particular, an exemplary operating environment for implementing technology described herein is shown and designated generally as exemplary operating environment 100. The exemplary operating environment 100 is but one example of a suitable computing environment and is not intended to suggest any limitation as to the scope of use or functionality of aspects of the technology described herein. Neither should the exemplary operating environment 100 be interpreted as having any dependency or requirement relating to any one component nor any combination of components illustrated.

Turning now to FIG. 1, a block diagram is provided showing an example operating environment 100 in which some aspects of the present disclosure may be employed. It should be understood that this and other arrangements described herein are set forth only as examples. Other arrangements and elements (e.g., machines, interfaces, functions, orders, and groupings of functions, etc.) can be used in addition to or instead of those shown, and some elements may be omitted altogether for the sake of clarity. Further, many of the elements described herein are functional entities that may be implemented as discrete or distributed components or in conjunction with other components, and in any suitable combination and location. Various functions described herein as being performed by one or more entities may be carried out by hardware, firmware, and/or software. For instance, some functions may be carried out by a processor executing instructions stored in memory.

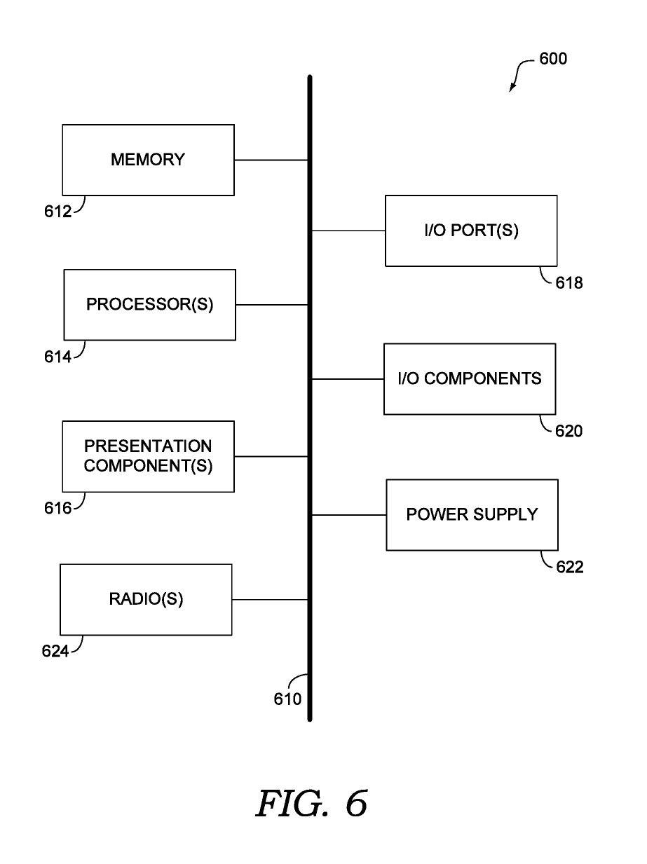

Among other components not shown, example operating environment 100 includes a number of user devices, such as user devices 102a and 102b through 102n; a number of data sources, such as data sources 104a and 104b through 104n; server 106; and network 110. It should be understood that environment 100 shown in FIG. 1 is an example of one suitable operating environment. Each of the components shown in FIG. 1 may be implemented via any type of computing device, such as computing device 600 described in connection to FIG. 6, for example. These components may communicate with each other via network 110, which may include, without limitation, one or more local area networks (LANs) and/or wide area networks (WANs). In exemplary implementations, network 110 comprises the Internet and/or a cellular network, amongst any of a variety of possible public and/or private networks.

User devices 102a and 102b through 102n can be client devices on the client-side of operating environment 100, while server 106 can be on the server-side of operating environment 100. The user devices can facilitate the completion of tasks and make a record of user activities. The user activities can be analyzed to determine when an exercise event has been performed by the user. Server 106 can comprise server-side software designed to work in conjunction with client-side software on user devices 102a and 102b through 102n so as to implement any combination of the features and functionalities discussed in the present disclosure. For example, the server 106 may run an exercise pattern inference engine 260, which identifies an exercise pattern for a user. The server 106 may receive activity records, such as physiological data and location data, from the user devices. This division of operating environment 100 is provided to illustrate one example of a suitable environment, and there is no requirement for each implementation that any combination of server 106 and user devices 102a and 102b through 102n remain as separate entities.

User devices 102a and 102b through 102n may comprise any type of computing device capable of use by a user. For example, in one aspect, user devices 102a through 102n may be the type of computing device described in relation to FIG. 6 herein. By way of example and not limitation, a user device may be embodied as a personal computer (PC), a laptop computer, a mobile device, a smartphone, a tablet computer, a smart watch, a wearable computer, a fitness tracker, a virtual reality headset, augmented reality glasses, a personal digital assistant (PDA), an MP3 player, a global positioning system (GPS) or device, a video player, a handheld communications device, a gaming device or system, an entertainment system, a vehicle computer system, an embedded system controller, a remote control, an appliance, a consumer electronic device, a workstation, or any combination of these delineated devices, or any other suitable device.

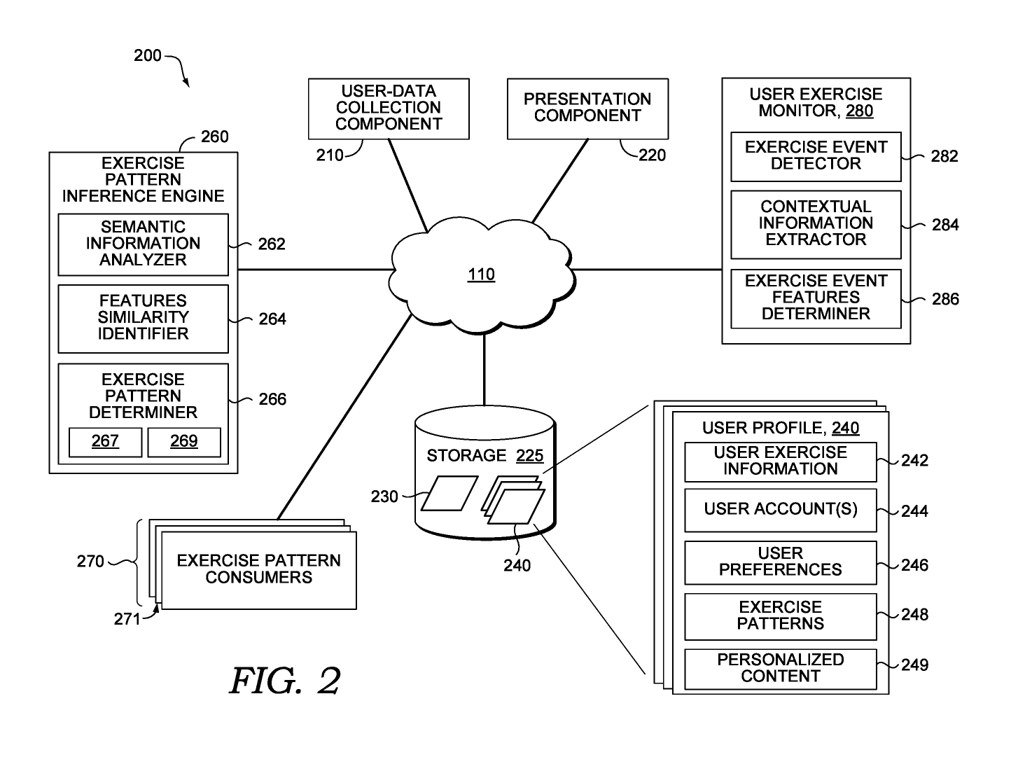

Data sources 104a and 104b through 104n may comprise data sources and/or data systems, which are configured to make data available to any of the various constituents of operating environment 100, or system 200 described in connection to FIG. 2. (For example, in one aspect, one or more data sources 104a through 104n provide (or make available for accessing) user data to user-data collection component 210 of FIG. 2.) Data sources 104a and 104b through 104n may be discrete from user devices 102a and 102b through 102n and server 106 or may be incorporated and/or integrated into at least one of those components. In one aspect, one or more of data sources 104a though 104n comprise one or more sensors, which may be integrated into or associated with one or more of the user device(s) 102a, 102b, or 102n or server 106. Examples of sensed user data made available by data sources 104a though 104n are described further in connection to user-data collection component 210 of FIG. 2. The data sources 104a though 104n can comprise a knowledge base that stores information about a venue, a user, or other entity related to a particular user action.

Operating environment 100 can be utilized to implement one or more of the components of system 200, described in FIG. 2, including components for collecting user data, identifying exercise events, inferring exercise patterns, and predicting probable exercise events in the future.

Referring now to FIG. 2, with FIG. 1, a block diagram is provided showing aspects of an example computing system architecture suitable for implementing an embodiment and designated generally as system 200. System 200 represents only one example of a suitable computing system architecture. Other arrangements and elements can be used in addition to or instead of those shown, and some elements may be omitted altogether for the sake of clarity. Further, as with operating environment 100, many of the elements described herein are functional entities that may be implemented as discrete or distributed components or in conjunction with other components, and in any suitable combination and location.

Example system 200 includes network 110, which is described in connection to FIG. 1, and which communicatively couples components of system 200 including user-data collection component 210, presentation component 220, user exercise monitor 280, exercise pattern inference engine 260, exercise pattern consumers 270, and storage 225. User exercise monitor 280 (including its components 282, 284, and 286), exercise pattern inference engine 260 (including its components 262, 264, 266, 267, and 269), user-data collection component 210, presentation component 220, and exercise pattern consumers 270 may be embodied as a set of compiled computer instructions or functions, program modules, computer software services, or an arrangement of processes carried out on one or more computer systems, such as computing device 600 described in connection to FIG. 6, for example.

In one embodiment, the functions performed by components of system 200 are associated with one or more personal assistant applications, services, or routines. In particular, such applications, services, or routines may operate on one or more user devices (such as user device 102a), servers (such as server 106), may be distributed across one or more user devices and servers, or be implemented in the cloud. Moreover, in some embodiments, these components of system 200 may be distributed across a network, including one or more servers (such as server 106) and client devices (such as user device 102a), in the cloud, or may reside on a user device, such as user device 102a. Moreover, these components, functions performed by these components, or services carried out by these components may be implemented at appropriate abstraction layer(s) such as the operating system layer, application layer, hardware layer, etc., of the computing system(s). Alternatively, or in addition, the functionality of these components and/or the embodiments described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. Additionally, although functionality is described herein with regards to specific components shown in example system 200, it is contemplated that in some embodiments functionality of these components can be shared or distributed across other components.

Continuing with FIG. 2, user-data collection component 210 is generally responsible for accessing or receiving (and in some cases also identifying) user data from one or more data sources, such as data sources 104a and 104b through 104n of FIG. 1. In some embodiments, user-data collection component 210 may be employed to facilitate the accumulation of user data of a particular user (or in some cases, a plurality of users including crowd-sourced data) for user exercise monitor 280, exercise pattern inference engine 260, or an exercise pattern consumer 270. The data may be received (or accessed), and optionally accumulated, reformatted and/or combined, by data collection component 210 and stored in one or more data stores such as storage 225, where it may be available to other components of system 200. For example, the user data may be stored in or associated with a user profile 240, as described herein. In some embodiments, any personally identifying data (i.e. user data that specifically identifies particular users) is either not uploaded or otherwise provided from the one or more data sources with user data, is not permanently stored, and/or is not made available to user exercise monitor 280 and/or exercise pattern inference engine 260.

User data may be received from a variety of sources where the data may be available in a variety of formats. For example, in some embodiments, user data received via user-data collection component 210 may be determined via one or more sensors, which may be on or associated with one or more user devices (such as user device 102a), servers (such as server 106), and/or other computing devices. As used herein, a sensor may include a function, routine, component, or combination thereof for sensing, detecting, or otherwise obtaining information such as user data from a data source 104a, and may be embodied as hardware, software, or both. By way of example and not limitation, user data may include data that is sensed or determined from one or more sensors (referred to herein as sensor data), such as location information of mobile device(s), properties or characteristics of the user device(s) (such as device state, charging data, date/time, or other information derived from a user device such as a mobile device), user-activity information (for example: app usage; online activity; searches; voice data such as automatic speech recognition; activity logs; communications data including calls, texts, instant messages, and emails; website posts; other user-data associated with communication events; etc.) including, in some embodiments, user activity that occurs over more than one user device, user history, session logs, application data, contacts data, calendar and schedule data, notification data, social-network data, news (including popular or trending items on search engines or social networks), online gaming data, ecommerce activity (including data from online accounts such as Microsoft®, Amazon.com®, Google®, eBay®, PayPal®, video-streaming services, gaming services, or Xbox Live®), user-account(s) data (which may include data from user preferences or settings associated with a personal assistant application or service), home-sensor data, appliance data, global positioning system (GPS) data, vehicle signal data, traffic data, weather data (including forecasts), wearable device data (which may include physiological data about the user such as heart rate, pulse oximeter or blood oxygen level, blood pressure, galvanic skin response, or other physiological data capable of being sensed or detected, other user device data (which may include device settings, profiles, network-related information (e.g., network name or ID, domain information, workgroup information, connection data, Wi-Fi network data, or configuration data, data regarding the model number, firmware, or equipment, device pairings, such as where a user has a mobile phone paired with a Bluetooth headset, for example, or other network-related information), gyroscope data, accelerometer data, payment or credit card usage data (which may include information from a user's PayPal account), purchase history data (such as information from a user's Xbox Live, Amazon.com, or eBay account), other sensor data that may be sensed or otherwise detected by a sensor (or other detector) component(s) including data derived from a sensor component associated with the user (including location, motion, orientation, position, user-access, user-activity, network-access, user-device-charging, or other data that is capable of being provided by one or more sensor component), data derived based on other data (for example, location data that can be derived from Wi-Fi, Cellular network, or IP address data), and nearly any other source of data that may be sensed or determined as described herein.

In some respects, user data may be provided in user-data streams or signals. A “user signal” can be a feed or stream of user data from a corresponding data source. For example, a user signal could be from a smartphone, a home-sensor device, a GPS device (e.g., for location coordinates), a vehicle-sensor device, a wearable device, a user device, a gyroscope sensor, an accelerometer sensor, a calendar service, an email account, a credit card account, or other data sources. In some embodiments, user-data collection component 210 receives or accesses data continuously, periodically, or as needed.

User exercise monitor 280 is generally responsible for monitoring user data for information that may be used for identifying and defining user exercise events and patterns, which may include identifying and/or tracking features (sometimes referred to herein as “variables”) or other information regarding specific user actions and related contextual information. Embodiments of user exercise monitor 280 may determine, from the monitored user data, when the user participates in an exercise event. As described previously, the user exercise information determined by user exercise monitor 280 may include user exercise information from multiple user devices associated with the user (e.g., fitness tracker and mobile phone) and/or from cloud-based services associated with the user (such as email, calendars, social-media, or similar information sources), and which may include contextual information associated with the identified user exercise. User exercise monitor 280 may identify current or near-real-time user exercise information and may also identify historical user exercise information, in some embodiments, which may be determined based on gathering observations of user exercise over time, accessing user logs of past exercise (such as an exercise event data store). Further, in some embodiments, user exercise monitor 280 may identify user exercise events (which may include historical exercise) from other similar users (i.e. crowdsourcing), as described previously. For example, physiological information from other users co-located with the user during a potential exercise event may be analyzed to determine the exercise event was a golf match.

In some embodiments, information determined by user exercise monitor 280 may be provided to exercise pattern inference engine 260 including information regarding the current context and historical visits (historical observations). Some embodiments may also provide user exercise information, such as probable upcoming exercise events, to one or more exercise pattern consumers 270. As described previously, user exercise features may be determined by monitoring user data received from user-data collection component 210. In some embodiments, the user data and/or information about the exercise event determined from the user data is stored in a user profile, such as user profile 240.

In an embodiment, user exercise monitor 280 comprises one or more applications or services that analyze information detected via one or more user devices used by the user and/or cloud-based services associated with the user, to determine exercise information and related contextual information. Information about user devices associated with a user may be determined from the user data made available via user-data collection component 210, and maybe provided to exercise pattern inference engine 260, among other components of system 200.

As shown in example system 200, user exercise monitor 280 comprises a user exercise detector 282, contextual information extractor 284, and an exercise features determiner 286. In some embodiments, user exercise monitor 280, one or more of its subcomponents, or other components of system 200, such as exercise pattern consumers 270 or exercise pattern inference engine 260, may determine interpretive data from received user data. Interpretive data corresponds to data utilized by these components of system 200 or subcomponents of user exercise monitor 280 to interpret user data. For example, interpretive data can be used to provide other context to user data, which can support determinations or inferences made by the components or subcomponents. Moreover, it is contemplated that embodiments of user exercise monitor 280, its subcomponents, and other components of system 200 may use user data and/or user data in combination with interpretive data for carrying out the objectives of the subcomponents described herein. Additionally, although several examples of how user exercise monitor 280 and its subcomponents may identify user exercise information are described herein, many variations of user exercise identification and user exercise monitoring are possible in various embodiments of the disclosure.

User exercise detector 282, in general, is responsible for determining (or identifying) that an exercise event has occurred. Embodiments of exercise detector 282 may be used for determining current exercise events and/or historical exercise events. Some embodiments of exercise detector 282 may monitor user data for exercise-related features or variables corresponding to exercise events such as indications of visits to exercise centric venues, physiological data, calendar data, and communications mentioning exercise events.

Additionally, some embodiments of user exercise detector 282 extract from the user data information about use exercise events, which may include current user exercise, historical user exercise, and/or related information such as contextual information. (Alternatively or in addition, in some embodiments contextual information extractor 284 determines and extracts contextual information that is related to one or more exercise events. Similarly, in some embodiments, exercise features determiner 286 extracts information about user exercise, such exercise event related features, based on an identification of the exercise event determined by user exercise detector 282.) Examples of extracted user exercise information may include physiological information, location information, movement information, weather data, etc.), or nearly any other data related to exercise events. Among other components of system 200, the extracted exercise event information determined by exercise detector 282 may be provided to other subcomponents of user exercise monitor 280, exercise pattern inference engine 260, or one or more exercise pattern consumers 270. Further, the extracted exercise event information may be stored in a user profile associated with the user, such as in user exercise information component 242 of user profile 240. In some embodiments, exercise event detector 282 or user exercise monitor 280 (or its other sub components) performs conflation on the detected user exercise information. For example, overlapping information may be merged and duplicated or redundant information eliminated.

In some embodiments, the user exercise-related features may be interpreted to determine an exercise event has occurred. For example, in some embodiments, exercise detector 282 employs user exercise logic, which may include rules, conditions, to associations, to identify or classify user exercise. The classifying of exercise events can be based on feature-matching or determining similarity in features, which falls under pattern recognition. This type of classification may use pattern recognition, fuzzy logic, neural network, finite state machine, support vector machine, logistic regression, clustering, or machine learning techniques, similar statistical classification processes or, combinations of these to identify exercise events from user data. For example, exercise logic may specify types of physiological information that is associated with an exercise event, such as a user's heart rate staying a threshold amount above a baseline for a designated duration, in combination with location or movement data. Different patterns of activity may be mapped to different exercise events. For example, running, cycling, swimming, golf, tennis, and soccer may all have different activity patterns.

In some embodiments a user may specify features used for detecting an exercise event or even a specific type of exercise event. For example, upon detecting a possible exercise event, a personal assistant application may ask the user to confirm that she just worked out and/or ask the user what activity the user completed. Based on this feedback, activity patterns can be learned for the user and used to identify future exercise events. Similarly exercise event patterns from other users can be used to recognize exercise events for a particular user.

Negative patterns associated with non-exercise activities, such as mowing the lawn, shopping, etc. may be provided to help distinguish between exercise events and non-exercise events that require an exertion. In this way, the exercise logic may be used to distinguish genuine user exercise, or exercise that is valuable towards a user's fitness program or goals, from non-exercise related activities or activity that the user may perceive as exercise but is negligible. Once an exercise event is determined, these features or additional related features may be detected and associated with the detected exercise for use in determining exercise patterns.

In some embodiments, user exercise detector 282 runs on or in association with each user device for a user. Exercise detector 282 may include functionality that polls or analyzes aspects of the user device to determine user exercise related features, such as sensor output, applications running (and in some instances the content of the applications), network communications, and/or other user actions detectable via the user device including sequences of actions.

Contextual information extractor 284, in general, is responsible for determining contextual information related to the exercise events (detected by user exercise detector 282 or user exercise monitor 280), such as context features or variables associated with an exercise event, related information, and further responsible for associating the determined contextual information with the detected exercise event. In some embodiments, contextual information extractor 284 may associate the determined contextual information with the related exercise event and may also log the contextual information with the associated exercise event. Alternatively, the association or logging may be carried out by another service. For example, some embodiments of contextual information extractor 284 provide the determined contextual information to exercise features determiner 286, which determines exercise features of the exercise event and/or related contextual information.

Some embodiments of contextual information extractor 284 determine contextual information related to an exercise event such as entities related to the exercise (e.g., other people present during the exercise event) or the location or venue wherein the exercise event took place. By way of example and not limitation, this may include context features such as location data; which may be represented as a location stamp associated with the exercise event; contextual information about the location, such as venue information (e.g. this is the user's office location, home location, gym, etc.) time, day, and/or date, which may be represented as a timestamp associated with the exercise event; duration of the exercise event, other user exercise/activities preceding and/or following the exercise event, other information about the exercise such as entities associated with the exercise (e.g. venues, people, objects, etc.), information detected by sensor(s) on user devices associated with the user that is concurrent or substantially concurrent to the user exercise (e.g. motion information or physiological information detected on a fitness tracking user device), or any other information related to the user exercise that is detectable that may be used for determining patterns of user exercise. Other contextual information can include the amount of sleep a user received in the last week, average variance of fall-asleep and wake-up times, times the user ate last, what they ate, etc.—basically any contextual information that may be potentially usable to determine that a user's performance varies from one situation to another based on the contextual information.

In embodiments using contextual information related to user devices, a user device may be identified by detecting and analyzing characteristics of the user device, such as device hardware, software such as operating system (OS), network-related characteristics, user accounts accessed via the device, and similar characteristics. For example, information about a user device may be determined using functionality of many operating systems to provide information about the hardware, OS version, network connection information, installed application, or the like. In some embodiments, a device name or identification (device ID) may be determined for each device associated with a user. This information about the identified user devices associated with a user may be stored in a user profile associated with the user, such as in user account(s) 244 of user profile 240. In an embodiment, the user devices may be polled, interrogated, or otherwise analyzed to determine contextual information about the devices. This information may be used for determining a label or identification of the device (e.g. a device id) so that contextual information about an exercise event captured on one device may be recognized and distinguished from data captured by another user device. In some embodiments, users may declare or register a user device, such as by logging into an account via the device, installing an application on the device, connecting to an online service that interrogates the device, or otherwise providing information about the device to an application or service. In some embodiments devices that sign into an account associated with the user, such as a Microsoft® account or Net Passport, email account, social network, or the like, are identified and determined to be associated with the user.

In some implementations, contextual information extractor 284 may receive user data from user-data collection component 210, parse the data, in some instances, and identify and extract context features or variables (which may also be carried out by exercise features determiner 286). Context variables may be stored as a related set of contextual information associated with the exercise event, and may be stored in a user profile such as in user exercise information component 242. In some cases, contextual information may be used by one or more exercise pattern consumers, such as for personalizing content or a user experience, such as when, where, or how to present content. Contextual information also may be determined from the user data of one or more users, in some embodiments, which may be provided by user-data collection component 210 in lieu of or in addition to user exercise information for the particular user.

Exercise features determiner 286 is generally responsible for determining exercise-related features (or variables) associated with the exercise event that may be used for identifying patterns of user exercise. Exercise features may be determined from information about an exercise event and/or from related contextual information. In some embodiments, exercise features determiner 286 receives user-exercise or related information from user exercise monitor 280 (or its subcomponents), and analyzes the received information to determine a set of zero or more features associated with the exercise event. Common features for different events can be used to help establish an exercise pattern.

Examples of exercise-related features include, without limitation, location-related features, such as location of the user device(s) during the exercise event, venue-related information associated with the location, or other location-related information; time related features, such as time(s) of day(s), day of week or month the user exercise, or the duration of the exercise, or related duration information such as how long the user used an application associated with the exercise; user device-related features, such as device type (e.g. desktop, tablet, mobile phone, fitness tracker, heart rate monitor, etc.) hardware properties or profiles, OS or firmware properties, device Ds or model numbers, network-related information (e.g. mac address, network name, IP address, domain, work group, information about other devices detected on the local network, router information, proxy or VPN information, other network connection information, etc.), position/motion/orientation related information about the user device, power information such as battery level, time of connecting/disconnecting a charger, user-access/touch information; usage related features, such as file(s) accessed, app usage (which may also include application data, in-app usage, concurrently running applications), network usage information, online activity (e.g. exercise related searches, browsed exercise websites, purchases, social networking related to exercise, communications sent or received including social media posts, user account(s) accessed or otherwise used, (such as device account(s), OS level account(s), or online/cloud-services related account(s) activity, such as Microsoft® account or Net Passport, online storage account(s), email, calendar, or social networking accounts, etc.), features that may be detected concurrent with the exercise event or near the time or the exercise event, or any other features that may be detected or sensed and used for determining a pattern of user exercise. Features may also include information about user(s) using the device; other information identifying a user, such as a login password, biometric data, which may be provided by a fitness tracker or biometric scanner; and/or characteristics of the user(s) who use the device, which may be useful for distinguishing users on devices that are shared by more than one user. In some embodiments, exercise inference logic 230 (described in connection to user exercise detector 282) may be utilized to identify specific features from user exercise information.

Continuing with system 200 of FIG. 2, exercise pattern inference engine 260 is generally responsible for determining user exercise patterns based on the exercise event information determined from user exercise monitor 280. In some embodiments, exercise pattern inference engine 260 may run on a server, as a distributed application across multiple devices, or in the cloud. At a high level, exercise pattern inference engine 260 may receive user-exercise-related information, which may be uploaded from user-exercise logs from client-side applications or services associated with user exercise monitor 280. One or more inference algorithms may be applied to the user-exercise-related information to determine a set of likely user exercise patterns. For example, patterns may be determined based on similar instances of observation of exercise events or associated contextual information, which may be referred to as “in-common features” of user exercise-related information. The inferred exercise pattern information may be provided to an exercise pattern consumer 270 and/or used to generate a pattern based prediction regarding likely future user exercise event(s). In some embodiments, a corresponding confidence is also determined for the patterns (or predictions based on the patterns), as described previously. Further, the exercise pattern (or prediction of future exercise based on a pattern) may comprise a single future (future-occurring) exercise event to occur, a sequence of exercise events, or probabilities for more than one future exercise event; for example, an eighty percent likelihood that the next exercise event will be running for 30 minutes on Monday and a fifteen percent likelihood that the next exercise event will be cycling on Monday, and a five percent likelihood that the next exercise event will be going to the gym on Tuesday.

As shown in example system 200, exercise pattern inference engine 260 comprises semantic information analyzer 262, features similarity identifier 264, and exercise pattern determiner 266. Semantic information analyzer 262 is generally responsible for determining semantic information associated with the exercise event related features identified by user exercise monitor 280. For example, while an exercise event feature may indicate a specific type of exercise (e.g., tennis) the semantic analysis may determine the tennis club the user belongs to, playing partners at the club, upcoming tennis tournaments the user has registered for or other entities associated with the exercise event. Semantic information analyzer 262 may determine an additional exercise event related features semantically related to the exercise event that may be used for identifying user exercise patterns. For example, the user plays tennis twice as often in the two weeks before a tournament.

In particular, as described previously, a semantic analysis is performed on the user exercise information, which may include contextual information, to characterize aspects of the user actions or exercise event. For example, exercise features associated with an exercise event may be categorized (such as by type, timeframe or location, work-related, home-related, themes, related entities, other user(s) (such as communication to or from another user about an exercise event) and/or relation of the other user to the user (e.g. family member, close friend, work acquaintance, boss, team member, club member, or the like), or other categories), or related features may be identified for use in determining a similarity or relational proximity to other user exercise events, which may indicate a pattern. In some embodiments, semantic information analyzer 262 may utilize a semantic knowledge representation, such as a relational knowledge graph. Semantic information analyzer 262 may also utilize semantic analysis logic, including rules, conditions, or associations to determine semantic information related to the user exercise. For example, a user exercise event comprising playing a sport with someone who works with the user may be characterized as a work-related exercise. Thus, where the user plays a sport with someone she works, but not necessarily the same person, every Sunday night, a pattern may be determined (using exercise pattern determiner 266) that the user plays in a work related sports league every Sunday night. Accordingly, it may be appropriate to surface a notification to the user, such as a reminder relating to the user's upcoming event on Sunday night since the user has a pattern of exercising on Sunday night. (Here, the notification service is one example of an exercise pattern consumer 270).

Semantic information analyzer 262 may also be used to characterize contextual information associated with the exercise event, such as determining that a location associated with the exercise event corresponds to a venue of interest to an exercise pattern (such as the user's home, work, gym, or the like) based on frequency of user visits. For example, the user's home hub may be determined (using semantic analysis logic) to be the location where the user spends most of her time between 8 PM and 6 AM.) Similarly, the semantic analysis may determine time of day that correspond to working hours, lunch time, commute time, etc. Different exercise patterns may be related to these designations. For example, a user may not exercise during work hours, except during lunch time.

Feature similarity identifier 264 is generally responsible for determining similarity of exercise features of user exercise events (put another way, exercise features characterizing a first user exercise event that are similar to exercise features characterizing a second user exercise event). The exercise features may include features relating to contextual information and features determined by semantic information analyzer 262. Exercise events having in-common exercise features may be used to identify an exercise pattern or sub pattern, such as by exercise pattern determiner 266.

For example, in some embodiments, features similarity identifier 264 may be used in conjunction with exercise pattern determiner 266 to determine a set of user exercise events that have in-common features. In some embodiments, this set of user exercise events may be used as inputs to a pattern based predictor, as described below. In some embodiments, features similarity identifier 264 comprises functionality for determining similarity of periodic- and behavioral-based exercise features. Periodic features comprise, for example, features that may occur periodically; for example, exercise events occurring on a day of the week or month, even/odd days (or weeks), monthly, yearly, every other day, every 3^{rd} day, etc. Behavior features may comprise behaviors such as user activities that tend to occur with certain locations or activities occurring before or after a given user exercise event (or sequence of previous exercise events), for example. For example, a user may exercise after a big meeting or every time the user is at a location, such as a park or gym.

In embodiments where exercise features have a value, similarity may be determined among different exercise features having the same value or approximately the same value, based on the particular feature. (For example, a timestamp of a first exercise happening at 12:01 on Friday and a timestamp of a second exercise happening at 12:07 on Friday may be determined to have similar or in-common timestamp features.)

Exercise pattern determiner 266 is generally responsible for determining a user exercise pattern based on similarities identified in user exercise information. In particular, exercise pattern determiner 266 (or exercise pattern inference engine 260) may determine a user exercise pattern based on repetitions of similar exercise features associated with a plurality of observed exercise events. In some embodiments, exercise events or patterns may be determined using exercise inference logic 230, such as rules, associations, conditions, prediction models, or pattern inference algorithms. The exercise inference logic 230 can comprise the logic (rules, associations, statistical classifiers, etc.) used for identifying and classifying exercise events, and also for determining exercise patterns. The user exercise information may be received from user exercise monitor 280 and information about identified similar features may be received from features similarity identifier 264. In some embodiments, the user pattern(s) determined by exercise pattern determiner 266 may be stored as inferred exercise patterns data store 248 in user profile 240.

In some embodiments, exercise pattern determiner 266 provides a pattern of user exercise and an associated confidence score regarding the strength of the user pattern, which may reflect the likelihood that future user exercises will follow the pattern. More specifically, in some embodiments, a corresponding confidence weight or confidence score may be determined regarding a determined user exercise pattern. The confidence score may be based on the strength of the pattern, which may be determined by the number of observations (of a particular user exercise event) used to determine a pattern, how frequently the user's actions are consistent with the exercise pattern, the age or freshness of the exercise observations, the number of features in common with the exercise observations that make up the pattern, or similar measurements.

In some instances, the confidence score may be considered when providing a determined exercise pattern to an exercise pattern consumer 270. For example, in some embodiments, a minimum confidence score may be needed before using the exercise pattern to provide an improved user experience or other service by an exercise pattern consumer 270. In one embodiment, a threshold of 0.6 (or just over fifty percent) is utilized such that only exercise patterns having a 0.6 (or greater) likelihood of predicting user exercise may be may be provided. Nevertheless, where confidence scores and thresholds are used, determined patterns of user exercise with confidence scores less than the threshold may still be monitored and updated based on additional exercise observations, since the additional observations of may increase the confidence for a particular pattern.

Some embodiments of exercise pattern determiner 266 determines a pattern according to the example approaches described below, where each instance of a workout event (or a particular type of classified workout event), has corresponding historical values of tracked exercise features (variables) that form patterns, and exercise pattern determiner 266 may evaluate the distribution of the tracked variables for patterns. In the following example, a tracked variable for a user exercise event is a timestamp corresponding to an observed instance of the user workout event. However, it will be appreciated that, conceptually, the following can be applied to different types of historical values.

A bag of timestamps (i.e., values of a given tracked variable) can be denoted as {tₘ}ₘ₌₁^{M}, and mapped to a two-dimensional histogram of hours and days of the week. The two-dimensional histogram can comprise a summation over the instances of the workout event, such as:

hᵢⱼ=Σₘ₌₁^{M}I[dayOfWeek[tₘ]=i] [symbol] I[hourOfDay[tₘ]=j].

This histogram can be used to determine derivative histograms. For example, a day of the week histogram may correspond to: hⱼ=Σᵢhᵢⱼ. An hour of the day histogram may correspond to: hᵢ=Σⱼhᵢⱼ. As further examples, one or more histograms may be determined for particular semantic time resolutions in the form of: h_{iC}=Σ_{jεC}hᵢⱼ. Any of various semantic time resolutions may be employed, such as weekdays and weekends, or morning, afternoon, and night. An example of the latter is where Cε{morning,afternoon,night}, morning={9, 10, 11}, afternoon={12, 13, 14, 15, 16}, and night={21, 22, 23, 24}.

An additional data structure utilized in representing an event can comprise the number of distinct time stamps in every calendar week that has at least one timestamp therein, which may be represented as:

wᵢ^{j}=∥{m|tₘ is within the i−the j week period}∥.

As an example, w₂³ can denote the number of distinct timestamps during the 2^{nd} three-week period of available timestamps. N^{(j)} may be utilized to denote the number of j-week time stamps available in the tracked data; for example, N⁽³⁾ denotes the number of three-week periods available in the timestamps.

Exercise pattern determiner 266 (or exercise pattern inference engine 260) may generate a confidence score that quantifies a level of certainty that a particular pattern is formed by the historical values in the tracked variable. In the following example, the above principles are applied utilizing Bayesian statistics. In some implementations, a confidence score can be generated for a corresponding tracked variable that is indexed by a temporal interval of varying resolution. For timestamps, examples include Tuesday at 9 am, a weekday morning, and a Wednesday afternoon. The confidence score may be computed by applying a Dirchlet-multinomial model and computing the posterior predictive distribution of each period histogram. In doing so, a prediction for each bin in a particular histogram may be given by:

$${x}_{i} = \frac{{α}_{0} + {h}_{i}}{{\sum }_{i}^{K} ( {α}_{0} + {h}_{i} )} ;$$

where K denotes the number of bins, α₀ is a parameter encoding the strength of prior knowledge, and i*=arg maxᵢ xᵢ. Then, the pattern prediction is the bin of the histogram corresponding to i* and its confidence is given by xᵢ*. As an example, consider a histogram in which morning=3, afternoon=4, and evening=3. Using α₀=10, the pattern prediction is afternoon, and the confidence score is

$$\frac{10 + 4}{( 10 + 3 ) + ( 10 + 4 ) + ( 10 + 3 )} = \frac{14}{40} \approx 0.35 .$$

In accordance with various implementations, more observations result in an increased confidence score, indicating an increased confidence in the prediction. As an example, consider a histogram in which morning=3000, afternoon=4000, and evening=3000. Using a similar calculation, the confidence score is

$$\frac{4010}{10030} \approx 0.4 .$$

Also, in some implementations, a confidence score can be generated for a corresponding tracked variable that is indexed by a period and a number of timestamps. Examples include 1 specific exercise event per week, and 3 of the specific exercise events every 2 weeks. Using a Gaussian posterior, a confidence score may be generated for a pattern for every period resolution, denoted as j. This may be accomplished by employing the formula:

$$= λ ( \frac{1}{{N}^{( j )}} {\sum }_{i}^{{N}^{( j )}} {w}_{i}^{( j )} ) + ( 1 - λ ) {μ}_{0} , where λ = \frac{{σ}_{0}^{2}}{\frac{{σ}^{2}}{{N}^{( j )}} + {σ}_{0}^{2}} .$$

In the foregoing, σ² is the sample variance, and σ₀² and μ₀ are parameters to the formula. A confidence score can be computed by taking a fixed interval around the number of time stamps prediction and computing the cumulative density as:

$${conf}_{j} = P ( \vert x - \vert < a ) = {\int }_{- a}^{+ a} 𝒩 ( x \vert , {\hat{σ}}^{( j )} ) , \\ where {\hat{σ}}^{( j )} = \frac{1}{\frac{{N}^{( j )}}{{σ}^{2}} + \frac{1}{{σ}_{0}^{2}}} .$$

As an example, consider the following observations: w₁⁽¹⁾=10, w₂⁽¹⁾=1, w₃⁽¹⁾=10, w₄⁽¹⁾=0, w₁⁽²⁾=11, and w₂⁽²⁾=10. N⁽¹⁾=4 and N⁽²⁾=2. Using μ₀=1 and σ₀²=10, μ⁽¹⁾=4.075, and conf₁=0.25. Furthermore μ⁽²⁾=10.31 and conf₂=0.99. In the foregoing example, although fewer time stamps are available for two week periods, the reduced variance in the user signals results in an increased confidence that a pattern exists.

Having determined that a pattern exists, or that the confidence score for a pattern is sufficiently high (e.g., satisfies a threshold value), exercise pattern determiner 266 may identify that a plurality of exercise events for the user corresponds to a user exercise pattern for the user. As a further example, exercise pattern determiner 266 may determine that a workout exercise pattern is likely to be followed by a user where one or more of the confidence scores for one or more tracked variables satisfy a threshold value.

In some embodiments, patterns of user exercise events may be determined by monitoring one or more exercise features, as described previously. These monitored exercise features may be determined from the user data described previously as tracked variables or as described in connection to user-data collection component 210. In some cases, the variables can represent context and/or semantic similarities among multiple user actions (exercise events). In this way, patterns may be identified by detecting variables or features in common over multiple exercise events. More specifically, features associated with a first event may be correlated with features of a second event to determine a likely pattern. An identified feature pattern may become stronger (i.e., more likely or more predictable) the more often the workout event-related observations that make up the pattern are repeated. Similarly, specific features can become more strongly associated with a user exercise pattern as they are repeated.

In some embodiments, such as the example embodiment shown in system 200, exercise pattern determiner 266 includes one or more pattern-based predictors 267 and 269. While multiple pattern predictors may be used, only pattern-based predictor 267 is described herein in detail. Pattern-based predictor 269 could operate in a similar manner, but perhaps operate on a different pattern when a user is associated with multiple patterns. Pattern-based predictor 267 comprises one or more predictors for predicting a next or future use exercise event by the user based on patterns, such as behavior or similarity features. At a high level, a pattern-based predictor 267 receives user exercise information and/or associated exercise features and determines a prediction of the next exercise event or probable future exercise event. In an embodiment, a pattern-based predictor 267 includes functionality for performing user exercise filtering, determining an exercise score, selecting an exercise event based on the score, and determining a particular pattern-based prediction.

In one embodiment, a pattern-based predictor 267 uses features similarity identifier 264 to determine patterns or features in common between historical exercise events and a recent exercise event. For example, periodic features similarity may be determined, from among the set of historical exercise events, those historical events having a periodic feature in common with a current or recent user action. Thus for example, if the recent exercise event happened on a Monday on the first day of the month, on an even week, and on a week day, then determining periodic features similarity would identify those historical exercise events that have features indicating the exercise event happened on a Monday, those historical exercise events having features corresponding to first day of the month (any first day, not just Mondays), or an even week, or a week day. Likewise, behavior features similarity may be determined to identify sets of historical exercise events having a particular behavior feature in common with a current or recent exercise event.

User exercise filtering may use the feature similarity determinations to filter out historical exercise events and retain only those historical exercise events that have a particular feature (or features) in common with a current or recent exercise event. Thus, in some embodiments, each pattern-based predictor may be designed (or tuned) for determining a prediction based on a particular feature (or features); for example there might be a set of predictors used for determining a prediction when the feature indicates a work day, or weekend, or Monday, or particular type of exercise event, etc. Such a predictor needs only those historical exercise events corresponding to its prediction model. (In some embodiments, such predictors may utilize specific prediction algorithms or models, based on their type of pattern prediction (prediction model). These algorithms or models may be stored in with exercise inference logic 230 in storage 225.)

A prediction of the user's next action (or future action) may be inferred based on (or according to) the example set of historical exercise events. In an embodiment, the predicted next user exercise event (or probable future exercise event) is the next exercise event with the highest observations count (i.e. the next exercise event that is predicted the most based on the set of example exercise events). Those historical exercise events in the example user actions that are consistent with the prediction may comprise the “prediction support set.” In some embodiments, a prediction probability corresponding to the prediction may be determined, such as based on a ratio of the size of the prediction support set vs. the total number of observations (historical user exercise events in the subset determined by the user action filtering). Moreover, in some embodiments, the prediction may also comprise additional information about information related to the user act exercise event, such as exercise features which characterize the predicted exercise event. (For example, if the predicted exercise event is that the user will run on Monday, additional exercise features may indicate that user will run in the morning, the people the user will run with, or other related information.) These may also be determined based on the exercise features of the prediction support set observations. Some embodiments also determine a prediction significance, which may be determined based on a confidence interval (e.g. a Binomial confidence interval) or other statistical appropriate statistical measure. Still further, in some embodiments, the prediction confidence may be based on the prediction probability and the prediction significance (e.g. the product of the prediction probability and prediction significance). Accordingly, some embodiments of exercise pattern determiner 266 provide a predicted next (or future) exercise event or series of next (or future) exercise events for each of the pattern-based predictor 267.

Some embodiments determine a specific prediction from pattern-based predictor 267. In an embodiment, an ensemble process is utilized wherein the pattern-based predictor 267 vote, and a selection is determined based on the member predictors. Further, in some embodiments, ensemble-member predictors may be weighted based on learned information about the exercise events. In one embodiment, once each pattern-based predictor 267 has provided a prediction, the prediction that has the highest corresponding confidence is determined as the next (or future) predicted user action, and may be considered a pattern-based (or history-based) prediction. In some embodiments, the output of exercise pattern determiner 266 may be stored as inferred exercise patterns data store 248 in user profile 240, and in some embodiments may be provided to an exercise pattern consumer 270.

Continuing with FIG. 2, example system 200 includes one or more exercise pattern consumers 270, which comprise applications or services that consume exercise pattern information to provide improved user experiences. Examples of exercise pattern consumers 270 may include, without limitation, fitness monitoring and training services, content personalization services, device power and management services, and semantic location services, among other services.

In particular, a first example exercise pattern consumer 270 comprises content personalization services. In one embodiment, a content personalization engine 271 is provided to facilitate providing a personalized user experience. Thus content personalization engine 271 may be considered one example of an application or service (or set of applications or services) that may consume information about user exercise patterns, which may include predictions of future user actions as determined by implementations of the present disclosure.

At a high level, example content personalization engine 271 is responsible for generating and providing aspects of personalized user experiences, such as personalized content or tailored delivery of content to a user. The content may be provided to the user as a personalized notification (such as described in connection to presentation component 220), may be provided to an application or service of the user (such as a calendar or scheduling application), or may be provided as part of an API where it may be consumed by yet another application or service.

In one embodiment, the personalized content may include a notification, which may comprise information, a reminder, a recommendation, suggestion, request, communication-related data (e.g. an email, instant message), or includes similar content that may be provided to the user in a way that is personalized. For example, content may be provided at a time when the user would most likely desire to receive it, such as an exercise related notification provided at a time according to a user pattern indicating that the user is about to begin an exercise event, and not providing the content at a time when it is likely to be dismissed, ignored, or bothersome.

In some embodiments, content personalization engine 271 tailors content for a user to provide a personalized user experience. For example, content personalization engine 271 may generate a personalized notification to be presented to a user, which may be provided to presentation component 220. Alternatively, in other embodiments, content personalization engine 271 generates notification content and makes it available to presentation component 220, which determines when and how (i.e., what format) to present the notification based on user data, user exercise pattern information. (For example, if a user exercise pattern indicates the user is likely to be driving to work at a time when it is relevant to present the notification, it may be appropriate to provide the notification in an audio format. Similarly in other situations, a notification may be provided as an in-app or toast notification.) In some embodiments, other services or applications operating in conjunction with presentation component 220 determine or facilitate determining when and how to present personalized content. Personalized content may be stored in a user profile 240, such as in a personalized content component 249.

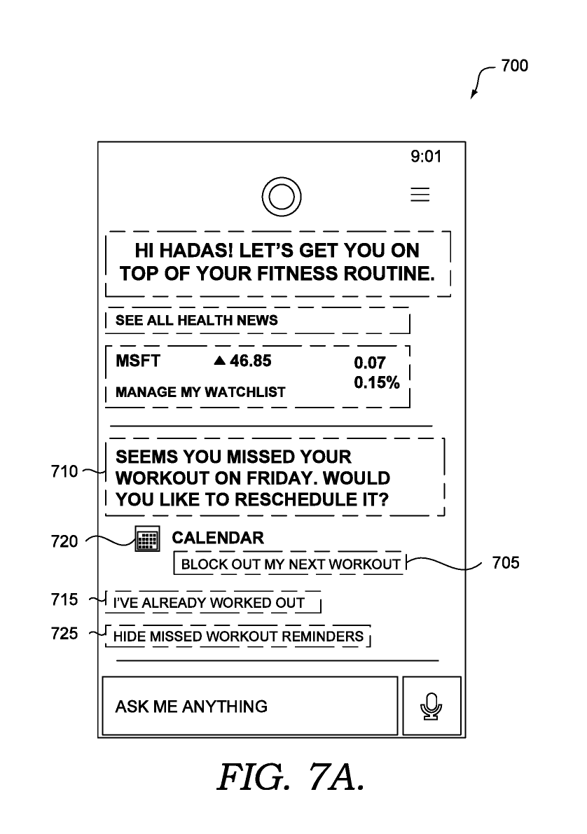

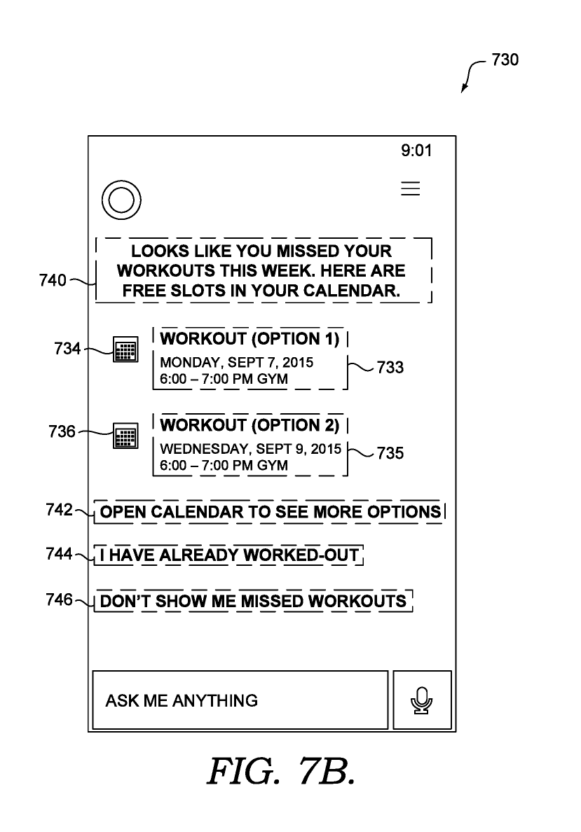

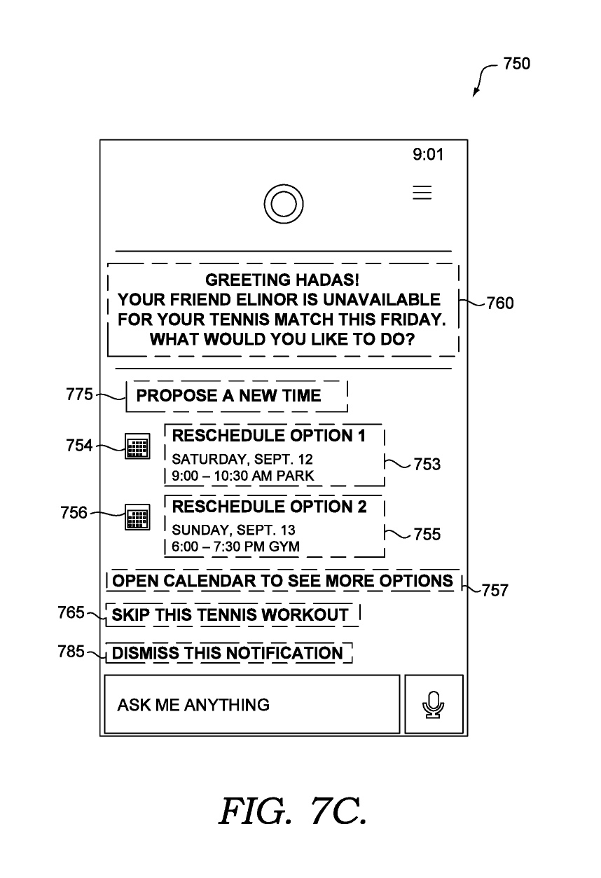

One type of service contemplated within some embodiments of this disclosure, and which may be facilitated using content personalization engine 271, comprises providing a notification to a user regarding a future or predicted workout event. For example, on the morning of the user's workout day, a notification may recommend that the user workout at a different location based on the weather forecast. For example, if it is likely that the user will run outside on a particular day, which may be determined based on an exercise pattern as described in connection to exercise pattern determiner 266, and contextual information indicates adverse weather conditions are forecasted at the time of the predicted future exercise event, then the user may be notified with a suggestion to move their exercise location indoors, to consider an alternate exercise activity, or reschedule it. In another example, if it is determined that the user likely missed a workout, according to a pattern of times when a user typically works out and may be expected to work out, then a notification may recommend a time to schedule a make-up workout. Turning briefly to FIG. 7A, one example of a user interface 700 for providing a notification for a missed workout is shown. Example user interface 700 includes a notification 710 (here, indicating that the user appears to have missed a workout, with a options for responding to the notification such as an optional response 715 enabling the user to indicate that she has already worked out and an optional response 725 enabling the user to request that these type or notifications (here, missed workout reminders) no longer be surfaced. Additionally, supplemental information may be provided to assist the user in responding to the notification, such as a link to the user's calendar 720. In some embodiments, an optional response 705 may be provided enabling the user to block out the next available time on the user's calendar for a make-up workout. Alternatively, user interface 700 may provide an optional response (not shown) suggesting one or more specific workout make-up times based on the user's availability and/or determined workout pattern(s); for example, if there is an opening in the user's calendar indicating availability later that day or the next day. Moreover, by combining user data from multiple user devices or sensors, and exercise pattern information, embodiments may provide a more accurate determination that a user has missed a workout or underperformed. For example, where it is determined that a user goes to a gym for an hour, it might appear that the user experienced a workout. But using other contextual information (e.g., scheduled calendar event, social media data, online activity), physiological data, or exercise pattern information it may be determined that the user did not workout at all but instead took his kids to the gym for a gymnastics class and worked on his computer during the class. Accordingly, a workout event would not be registered for the user, and in some embodiments, a notification such as shown in FIG. 7A may be surfaced to the user.

Another embodiment of a user interface 740 for providing a notification 745 to a user based on this missed workout event example is illustratively provided in FIG. 7B. This example user interface 730 includes a notification 740 indicating that it appears the user has missed one or more workouts. In this illustrative example, the user is presented with suggestions 733 (workout option 1) and 735 (workout option 2) for workout makeup times, based the user's availability. Links to the user's calendar for these times may also be provided, such as shown at items 734 and 736, as supplemental information, to assist the user in determining which workout option makeup time is more convenient. User interface 730 may also include other options for responding to notification 740, such as optional response 742 enabling the user to open her calendar to see more options for rescheduling the missed workout(s); optional response 744 enabling the user to indicate that she has already worked out; and optional response 746 enabling the user to request that these type or notifications (here, missed workout reminders) no longer be surfaced.

In yet another example and with reference now to FIG. 7C, where a user has a pattern of playing tennis, and it is determined that the user's usual opponent is unavailable, the user may be notified and recommended to reschedule her tennis game. FIG. 7C shows an example user interface 750 comprising a notification 760 indicating that the user's opponent (here, the user's friend Elinor) is unavailable. In some embodiments, the user may be presented with a time wherein the user and the opponent are both available, that is consistent with determined exercise patterns for both parties. This may be determined based on an exercise event pattern of playing tennis for the user and contextual information, such information about another person (who may identified using contact information of the user or social media friends who are usually co-located with the user at the time of tennis activity, or may be indicated in the user's calendar as a calendar event corresponding to the time(s) that the user plays tennis, for example. In the example depicted in FIG. 7C, user interface 750 includes suggested makeup times Reschedule Option 1 753 and Reschedule Option 2 755, which may also include links to the user's calendar for these specific proposed times (items 754 and 756) as supplemental information, to assist the user in determining which reschedule option is more convenient. Alternatively or in addition, user interface 750 may include a link to the user's calendar (757) enabling the user to see more options for rescheduling. In some embodiments, link 757 may include information from the calendars of both parties, enabling the user to more easily determine potential reschedule times when both parties are expected to be available. User interface 750 also includes response options 765, 775, and 785 enabling the user to simply skip this scheduled tennis workout (765), which may cause a notification to be sent to the other party, in some embodiments; propose a new tennis time based on a determined likely availability of both parties (775), and in some embodiments, based on available tennis court times; or dismiss the notification (785). In some embodiments, a response option (not shown) may be provided to automatically schedule (block out on both user's calendars) the suggested makeup time. Additionally, in some embodiments, other supplemental information may be provided to assist the user in responding to the notification, such as a link to information about available tennis court times and locations (not shown).

A second example exercise pattern consumer 270 comprises personalized fitness monitoring and training services, which may utilize or work in conjunction with content personalization engine 271. Examples of such services may comprise services for tracking contextual information associated with user workout events, identifying trends, patterns, or correlations with exercise performance or exercise quality and contextual information and providing to the user with this information or providing recommendations based on this information. For example, it may be determined that the user has a faster runtime on Tuesday's than on Saturdays, exercises longer or has better cardio readings when she runs at location X than at location Y, or burn more calories and sleeps better when she exercises with her friend Hadas then when she exercises by herself or with her friend Gal. Additionally, embodiments of such services may determine or create training and exercise goals that are personalized to the user based on learned contexts associated with the user and the user's exercise patterns. This might also include, for example, optimal exercise times, optimal exercise patterns, diet, exercise locations and location patterns (e.g. on a peak day it is recommended that the user works out at a location more likely to result in a higher-level of exercise, based on the user's exercise patterns or patterns from similar users.)

Still further, some embodiments of fitness monitoring and training services may personalize exercises for the user (such as recommendations, suggestions, goals, training regimens, etc.) based on exercise-related pattern information (including contextual information) from a population of similar users (i.e. crowdsourcing information). For example, in one embodiment that uses crowdsourcing, exercise pattern related information of other users semantically similar to a particular user may be used for determining additional features, which may be imputed onto the particular user's user device. Such similarities may include, without limitation, other users with similar workouts (e.g. long distance runners, boxers, golfers, etc.) living in the same region, exercising at the same location(s), having similar physiologies (including age, weight, sex, fitness level, etc.) or exercise routines, working together, eating similar diets, having similar daily schedules, or other similarities. In some embodiments, crowdsourcing may be employed for a new user or where little information is known about a particular user. For example, where a user who runs several days each week, and contextual information (e.g. determined from email, voicemail, doctor's visit, etc.), other sensor data (e.g. motion, gyroscopic, or acceleration information consistent with a foot injury, such as an unusual gait or limping) and/or physiological data from a wearable sensor user device indicates that the user has sustained an injury, a recommendation may be provided to the user regarding alternative workouts and/or therapies based on exercise-related information derived from similar users (other runners who have suffered an injury).

A third example exercise pattern consumer 270 comprises device power and management services. Examples of such services may include services that configure or manage device power, sensor power, sensor sensitivity, or otherwise manage one or more user devices or applications and services accessed using the user device(s). One such service comprises a device power management service that may determine when to activate a user device, such as a wearable fitness tracker, activate additional sensors on a user device, increase sensor sensitivity, or otherwise engage physical or virtual components on a user device (such as communication components, display components, processing, etc.) and therefore increase energy usage of the device. In particular, activation may be based according to a user's exercise pattern. For example, when a user's mobile phone (or other user device) detects the user is at the gym or determines that the current time corresponds to a time when the user typically has her workout, the mobile phone may communicate to a fitness tracker worn by the user to “wake it up” and begin a heightened monitoring state, such as turning on physiological sensors or taking readings on such sensors more often. If after a period of time, the wearable device does not detect workout activity, the device may resume a sleep state. In this way, the battery life of the wearable fitness tracker is preserved. Moreover, if the user does not need to remember to start or stop the active monitoring state of the fitness tracker, or otherwise declare a workout.

A fourth example exercise pattern consumer 270 comprises semantic location services. By way of example and not limitation, using user data (such as information provided from user-data collection component 210, which may include location data in some embodiments and not include location data in some embodiments) semantic location services such as venue identification and disambiguation may be provided. In one embodiment, where location data, which might be provided from a user's smartphone, indicates the user is either at a gym or an adjacent coffee house, data from a wearable fitness tracker or information about the user's exercise patterns may be used to disambiguate the venue. For example, where physiological data corresponds to an exercise event, it may be inferred that the user is at the gym and not the coffee house. Similarly, where the current time corresponds to a time when the user usually works out, it may be inferred that the user is more likely at the gym than at the coffee house. But where the current time corresponds to times when the user rarely or never exercises (such as 8:30 AM on a workday), then it may be inferred that the user is more likely at the coffee house, than at the gym. Additionally, where exercise events are repeatedly observed at a particular location, the location may be identified as an exercise venue. Some embodiments may consider information from other users as well. Thus where multiple users show exercise event activity at a particular location, the location may be identified as an exercise venue. In some embodiments, this venue may be recommended to users as a location for exercise.

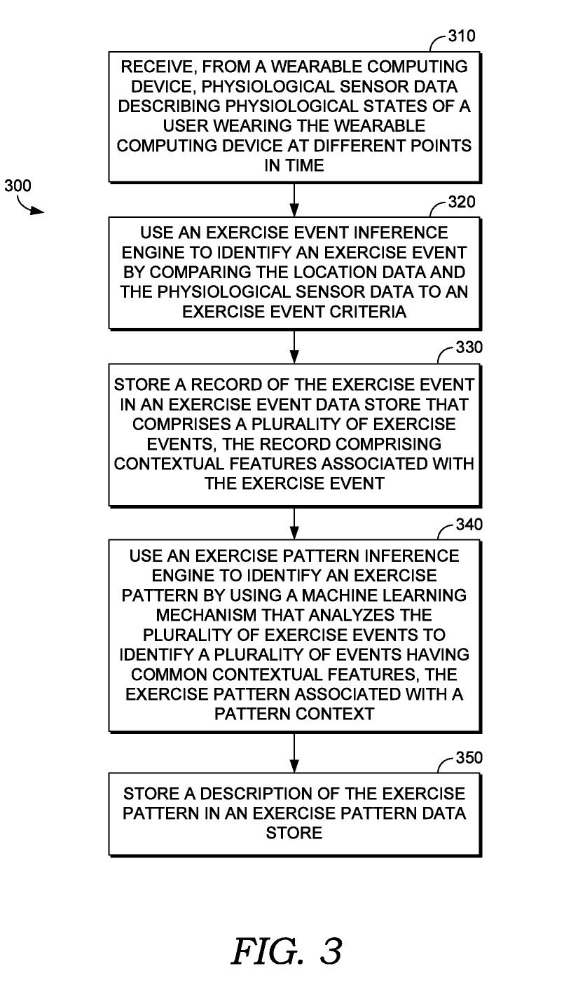

Turning now to FIG. 3, a flow diagram is illustrated showing an exemplary method 300 for a method of inferring an exercise pattern. Method 300 may be performed by one or more computing devices, such as a smart phone. The exercise pattern is inferred through the analysis of signal data gathered from multiple computing devices associated with the user.

At step 310 physiological sensor data is received from a wearable computing device. The physiological sensor data describes physiological states of a user wearing a wearable computing device. The sensor data can be time stamped to correlate the physiological state with different points in time. The physiological data can comprise heart rate, pulse oximeter or blood oxygen level, blood pressure, galvanic skin response, or other physiological data capable of being sensed or detected. The physiological sensor data may be communicated directly to a computing device performing a method 300. For example, the physiological signal data may be communicated from a wearable device to a smart phone over a wireless or wired connection. In another aspect, the physiological sensor data is retrieved from a data store associated with the user that stores physiological data gathered from one or more wearable computing devices. For example, a user may download physiological data to a laptop that uploads the physiological data to a cloud-based data store where it is retrieved by computing device performing method 300.

At step 320 an exercise event inference engine is used to identify an exercise event by comparing the location data and the physiological sensor data to exercise event criteria. The operation of an exercise inference engine has been described previously with reference to FIG. 2. The exercise criteria can take many different forms depending on the mechanism used to identify an exercise event. For example, the exercise event criteria could be training data used to train a neural network that is used to evaluate user data to determine when an exercise event has occurred. The classification of step 320 may use pattern recognition, fuzzy logic, neural network, finite state machine, support vector machine, logistic regression, clustering, or machine learning techniques, similar statistical classification processes or, combinations of these to identify exercise events from user data. For example, exercise logic may specify types of physiological information that is associated with an exercise event, such as a user's heart rate staying a threshold amount above a baseline for a designated duration, in combination with location or movement data. Different patterns of activity may be mapped to different exercise events.

At step 330, a record of the exercise event is stored in an exercise event data store that comprises a plurality of exercise events. The record comprises contextual features associated with the exercise event. The contextual features can include the specific type of exercise engaged in during the exercise event. In one aspect, different classifiers are used to identify different types of exercise events. For example user data could be fed to separate classifiers trained to identify gym workouts, cycling, soccer, and such.

At step 340 an exercise pattern inference engine is used to identify an exercise pattern by using a machine learning mechanism that analyzes the plurality of exercise events to identify a plurality of events having common contextual features. The exercise pattern is associated with a pattern context. The pattern context can include a periodic context and a behavioral context. The periodical context defines when the exercise pattern occurs, such as everyday, every other day, every Thursday, etc. The behavioral content may include other contextual features surrounding the exercise event.

At step 350 the exercise pattern is stored in an exercise pattern data store. The exercise pattern may be accessed by one or more computing applications that provide exercise related services to the user. The exercise pattern may be propagated to multiple computing devices associated with the user. A user may be associated with multiple exercise patterns. Each exercise pattern associated with the user may be stored in exercise pattern data store.

Once an exercise pattern is determined, it can be used to determine that a probable future exercise event will occur at a future time that is a threshold time from a present time (e.g. at or within three hours, twelve hours, one day, five days, two weeks, etc. from the current time) by analyzing the exercise pattern. The probable future exercise event can be associated with a location and other contextual data than can be used to facilitate user exercise related experiences. In one aspect, the additional contextual information can be a behavioral context, such as weather parameters when the exercise events occur. In addition, weather associated with exceptions to the pattern (i.e., when the user does not follow an established pattern by not exercising) can be compared to the weather when the exercise events occurred to define an acceptable weather range.

Continuing with the weather example, a weather forecast for the location at the future time can be determined and compared with the weather parameters. The weather forecast can be determined to not match a weather context (e.g., weather range) for the probable future exercise event. For example, the temperature may outside of the weather range, for example the forecast could be for a high of 30 degrees Fahrenheit, when the exercise range specifies a temperature between 45 and 90 degrees.

Upon detecting an exceptional circumstance suggesting the user will not follow the exercise pattern, a notification is generated for the user providing information about an alternative exercise venue that is inside. The notification can comprises an exercise class occurring at the alternative exercise venue during the future time.

The exercise pattern can be used to conserve energy consumed by the wearable computer device by automatically starting and stopping active monitoring. For example, a start instruction can be communicated to the wearable computer to start active tracking at the future time and a stop active tracking instruction can be communicated at a stop time that is calculated by adding an exercise duration to the future time. The exercise duration can be extracted the exercise pattern. The automated termination of active monitoring can save energy by stopping the active monitoring at an optimal time instead of whenever a user might stop the active monitoring.

The exercise pattern can be used to disambiguate a location. For example, at the future time the user may be present at a location associated with multiple venues. The exercise pattern could be used to determine that that user is present at a venue that facilitates exercise.

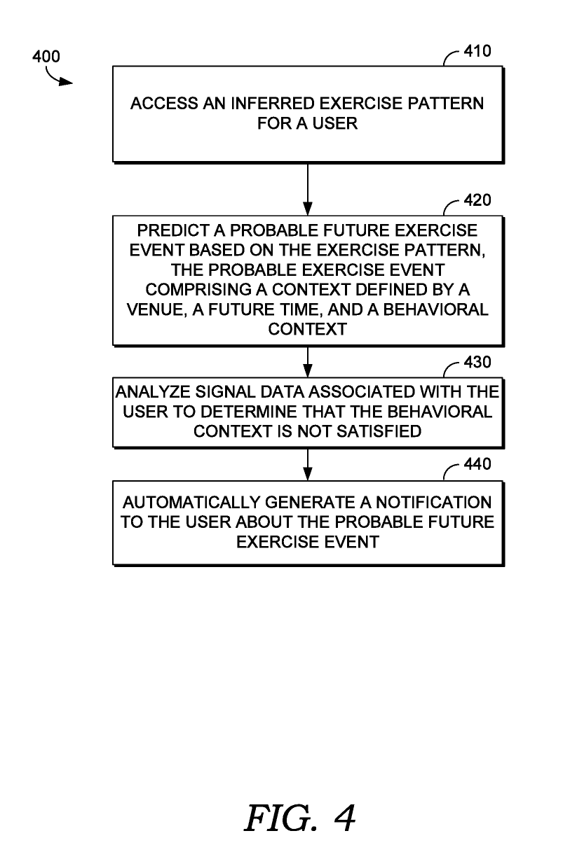

Turning now to FIG. 4, a method 400 of inferring an exercise pattern is provided.

At step 410 an inferred exercise pattern for a user is accessed. The inferred exercise pattern may be determined as described previously with reference to FIG. 2. As mentioned, a user may be associated with multiple exercise patterns. The pattern can be associated with various confidence levels that indicate how likely to pattern is to be followed.

At step 420 a probable future exercise event is predicted based on the exercise pattern. The probable future exercise event comprises a context defined by a venue, a future time, and a behavioral context. In one aspect, the probable future exercise event is determined looking only at periodic context. The periodic context defines when an exercise event associated with exercise pattern is likely to occur. For example, if the pattern indicates a user runs every Monday, Wednesday, and Friday then a future running event could be determined for any Monday, Wednesday, or Friday. Other contextual information may be evaluated based on behavioral context to determine whether or not the user is likely to follow the periodic context. The behavioral context defines contextual features present when the user keeps the periodic context. The behavioral context may also be defined in the negative to identify contextual features that define exceptions to the periodic context. For example, the user may not exercise on holidays.

At step 430 signal data associated with the user is analyzed to determine that the behavioral context is not satisfied.

At step 440 a notification to the user about the probable future exercise event is automatically generated. In one aspect, the notification is a communication interface having contact address for one or more of a plurality of people predicted to participate in the probable future exercise event. The notification can further comprises an auto generated message indicating the user will not be present at the probable future exercise event.

In one aspect, the behavioral context is an outdoor temperature within a specific range determined by analyzing previous exercise events involving an activity to be completed in the probable future exercise event. A forecast or present temperatures can be accessed to determine the weather conditions at the future time will not satisfy the behavioral context.

In one aspect, the notification comprises proposing an alternative exercise event time that corresponds with a predicted probable future exercise event time for one or more of the plurality of people that normally participate in the probable future exercise event.

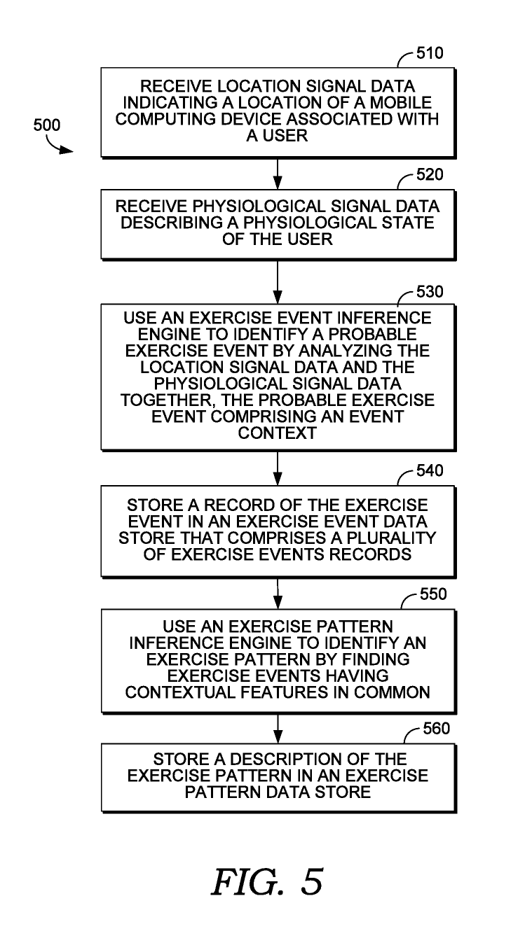

Turing now to FIG. 5, a method 500 of inferring an exercise pattern is provided.

At step 510 location signal data indicating a location of a mobile computing device associated with a user is received. In one aspect, method 500 is performed by a mobile computing device, such as a smart phone. In this circumstance, location signal data may be received from a component of the smart phone that generates location data.

At step 520 physiological signal data describing a physiological state of the user is received. The physiological signal data may be received from a wearable computing device, such as a fitness tracker. The physiological signal data may be received through a direct communication from a wearable computing device or indirectly through an intermediary computing device or devices.

At step 530 an exercise event inference engine is used to identify a probable exercise event by analyzing the location signal data and the physiological signal data together. The probable exercise event comprising an event context.

At step 540 a record of the exercise event is stored in an exercise event data store that comprises a plurality of exercise events records.

At step 550 an exercise pattern inference engine is used to identify an exercise pattern by finding exercise events having contextual features in common. The exercise pattern can be associated with a periodic context and a behavioral context.

At step 560 a description of the exercise pattern is stored in an exercise pattern data store. Aspects of the technology described herein can use the exercise pattern to detect exceptions to the pattern and provide user experiences around these exceptions. For example, the periodic context could indicate that a probable future exercise event will occur while the behavioral context is not satisfied. This pattern indicates that the user will not follow their established pattern in this particular instance. The behavioral context can include acceptable weather for the exercise event, being located in the user's home city, having a clear calendar around the scheduled exercise time, and such. When the behavioral context indicates that an exception to the exercise pattern will occur, the technology described herein can suggest an alternative exercise at an alternative time. The alternative suggestion can be based on an analysis of the user actions when previous exceptions occurred. In a sense, the exceptions for the user's actions when the main exercise pattern is not followed can form a separate sub exercise pattern. For example, when the user does not go on a scheduled run in the morning, observations of user actions can indicate that the user exercises at home in the evening on a treadmill.

In another aspect, the user may be associated with multiple exercise patterns. For example, a user may run three days a week and exercise at the gym on three different days. When the weather, or other behavioral context, suggest an exception will occur, the notification can provide a suggestion related to the other exercise pattern. For example, a notification could alert the user that rain is in the forecast on a run day and suggest that the user exercise at the gym instead. In addition to a notification, steps can be taken to help facilitate the rearrangement of the users exercise routine such as contacting other people that participate in the user's exercise events.

### Exemplary Operating Environment

Referring to the drawings in general, and initially to FIG. 6 in particular, an exemplary operating environment for implementing aspects of the technology described herein is shown and designated generally as computing device 600. Computing device 600 is but one example of a suitable computing environment and is not intended to suggest any limitation as to the scope of use of the technology described herein. Neither should the computing device 600 be interpreted as having any dependency or requirement relating to any one or combination of components illustrated.

The technology described herein may be described in the general context of computer code or machine-useable instructions, including computer-executable instructions such as program components, being executed by a computer or other machine, such as a personal data assistant or other handheld device. Generally, program components, including routines, programs, objects, components, data structures, and the like, refer to code that performs particular tasks or implements particular abstract data types. The technology described herein may be practiced in a variety of system configurations, including handheld devices, consumer electronics, general-purpose computers, specialty computing devices, etc. Aspects of the technology described herein may also be practiced in distributed computing environments where tasks are performed by remote-processing devices that are linked through a communications network.

With continued reference to FIG. 6, computing device 600 includes a bus 610 that directly or indirectly couples the following devices: memory 612, one or more processors 614, one or more presentation components 616, input/output (I/O) ports 618, I/O components 620, and an illustrative power supply 622. Bus 610 represents what may be one or more busses (such as an address bus, data bus, or a combination thereof). Although the various blocks of FIG. 6 are shown with lines for the sake of clarity, in reality, delineating various components is not so clear, and metaphorically, the lines would more accurately be grey and fuzzy. For example, one may consider a presentation component such as a display device to be an I/O component. Also, processors have memory. The inventors hereof recognize that such is the nature of the art and reiterate that the diagram of FIG. 6 is merely illustrative of an exemplary computing device that can be used in connection with one or more aspects of the technology described herein. Distinction is not made between such categories as “workstation,” “server,” “laptop,” “handheld device,” etc., as all are contemplated within the scope of FIG. 6 and refer to “computer” or “computing device.”

Computing device 600 typically includes a variety of computer-readable media. Computer-readable media can be any available media that can be accessed by computing device 600 and includes both volatile and nonvolatile media, removable and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules, or other data.

Computer storage media includes RAM, ROM, EEPROM, flash memory or other memory technology, CD-ROM, digital versatile disks (DVD) or other optical disk storage, magnetic cassettes, magnetic tape, magnetic disk storage or other magnetic storage devices. Computer storage media does not comprise a propagated data signal.

Communication media typically embodies computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, and other wireless media. Combinations of any of the above should also be included within the scope of computer-readable media.

Memory 612 includes computer storage media in the form of volatile and/or nonvolatile memory. The memory 612 may be removable, non-removable, or a combination thereof. Exemplary memory includes solid-state memory, hard drives, optical-disc drives, etc. Computing device 600 includes one or more processors 614 that read data from various entities such as bus 610, memory 612, or I/O components 620. Presentation component(s) 616 present data indications to a user or other device. Exemplary presentation components 616 include a display device, speaker, printing component, vibrating component, etc. I/O ports 618 allow computing device 600 to be logically coupled to other devices, including I/O components 620, some of which may be built in.

Illustrative I/O components include a microphone, joystick, game pad, satellite dish, scanner, printer, display device, wireless device, a controller (such as a stylus, a keyboard, and a mouse), a natural user interface (NUI), and the like. In aspects, a pen digitizer (not shown) and accompanying input instrument (also not shown but which may include, by way of example only, a pen or a stylus) are provided in order to digitally capture freehand user input. The connection between the pen digitizer and processor(s) 614 may be direct or via a coupling utilizing a serial port, parallel port, and/or other interface and/or system bus known in the art. Furthermore, the digitizer input component may be a component separated from an output component such as a display device, or in some aspects, the usable input area of a digitizer may coexist with the display area of a display device, be integrated with the display device, or may exist as a separate device overlaying or otherwise appended to a display device. Any and all such variations, and any combination thereof, are contemplated to be within the scope of aspects of the technology described herein.

An NUI processes air gestures, voice, or other physiological inputs generated by a user. Appropriate NUI inputs may be interpreted as ink strokes for presentation in association with the computing device 600. These requests may be transmitted to the appropriate network element for further processing. An NUI implements any combination of speech recognition, touch and stylus recognition, facial recognition, biometric recognition, gesture recognition both on screen and adjacent to the screen, air gestures, head and eye tracking, and touch recognition associated with displays on the computing device 600. The computing device 600 may be equipped with depth cameras, such as stereoscopic camera systems, infrared camera systems, RGB camera systems, and combinations of these, for gesture detection and recognition. Additionally, the computing device 600 may be equipped with accelerometers or gyroscopes that enable detection of motion. The output of the accelerometers or gyroscopes may be provided to the display of the computing device 600 to render immersive augmented reality or virtual reality.

A computing device may include a radio 624. The radio 624 transmits and receives radio communications. The computing device may be a wireless terminal adapted to receive communications and media over various wireless networks. Computing device 600 may communicate via wireless protocols, such as code division multiple access (“CDMA”), global system for mobiles (“GSM”), or time division multiple access (“TDMA”), as well as others, to communicate with other devices. The radio communications may be a short-range connection, a long-range connection, or a combination of both a short-range and a long-range wireless telecommunications connection. When we refer to “short” and “long” types of connections, we do not mean to refer to the spatial relation between two devices. Instead, we are generally referring to short range and long range as different categories, or types, of connections (i.e., a primary connection and a secondary connection). A short-range connection may include a Wi-Fi® connection to a device (e.g., mobile hotspot) that provides access to a wireless communications network, such as a WLAN connection using the 802.11 protocol. A Bluetooth connection to another computing device is a second example of a short-range connection. A long-range connection may include a connection using one or more of CDMA, GPRS, GSM, TDMA, and 802.16 protocols.

The technology described herein has been described in relation to particular aspects, which are intended in all respects to be illustrative rather than restrictive. While the technology described herein is susceptible to various modifications and alternative constructions, certain illustrated aspects thereof are shown in the drawings and have been described above in detail. It should be understood, however, that there is no intention to limit the technology described herein to the specific forms disclosed, but on the contrary, the intention is to cover all modifications, alternative constructions, and equivalents falling within the spirit and scope of the technology described herein.

What is claimed is:

1. A computing system comprising:
a processor;
one or more sensors configured to provide sensor data, including, at least location data for a mobile computing device; and
computer storage memory having computer-executable instructions stored thereon which, when executed by the processor, implement a method of inferring an exercise pattern, the method comprising:
(1) receiving, from a wearable computing device, physiological sensor data describing physiological states of a user wearing the wearable computing device at different points in time;
(2) using an exercise event inference engine to identify an exercise event by comparing the location data and the physiological sensor data to an exercise event criteria;
(3) storing a record of the exercise event in an exercise event data store that comprises a plurality of exercise events, the record comprising contextual features associated with the exercise event;
(4) using an exercise pattern inference engine to identify the exercise pattern by using a machine learning mechanism that analyzes the plurality of exercise events to identify a plurality of events having common contextual features, the exercise pattern associated with a pattern context; and
(5) storing a description of the exercise pattern in an exercise pattern data store.

2. The system of claim 1, wherein the method further comprises:
determining that a probable future exercise event will occur at a future time that is a threshold time from a present time by analyzing the exercise pattern, the probable future exercise event associated with a location;
determining a weather forecast for the location at the future time;
determining that the weather forecast does not match a weather context for the exercise pattern; and
generating a notification for the user providing information about an alternative exercise venue that is inside.

3. The system of claim 2, wherein the notification comprises an exercise class occurring at the alternative exercise venue during the future time.

4. The system of claim 1, wherein the pattern context includes a periodic context and a behavioral context.

5. The system of claim 2, wherein the method further comprises communicating a start instruction to the wearable computing device to start active tracking at the future time.

6. The system of claim 2, wherein the method further comprises communicating a stop instruction to the wearable computing device to stop active tracking a stop time that is calculated by adding an exercise duration to the future time, wherein the exercise duration is extracted from the exercise pattern.

7. The system of claim 2, wherein the method further comprises:
receiving location data at the future time;
determining that multiple venues are associated with the location data; and
determining that the user is in an exercise venue because the exercise pattern indicates an the probable future exercise event is occurring.

8. A method of inferring an exercise pattern, the method comprising:
accessing an inferred exercise pattern for a user;
predicting a probable future exercise event based on the exercise pattern, the probable future exercise event comprising a context defined by a venue, a future time, and a behavioral context; and
analyzing signal data associated with the user to determine that the behavioral context is not satisfied; and
automatically generating a notification to the user about the probable future exercise event.

9. The method of claim 8, wherein the notification is a communication interface having contact address for one or more of a plurality of people predicted to participate in the probable future exercise event.

10. The method of claim 9, wherein the notification further comprises an auto generated message indicating the user will not be present at the probable future exercise event.

11. The method of claim 8, wherein the behavioral context is an outdoor temperature within a specific range determined by analyzing previous exercise events involving an activity to be completed in the probable future exercise event.

12. The method of claim 8, wherein the notification comprises proposing an alternative exercise event time that corresponds with a predicted probable future exercise event time for one or more of a plurality of people that are associated with predicted probable future exercise event.

13. The method of claim 8, wherein the method further comprise generating a plurality of exercise events for the user by analyzing location data generated by a mobile user device and physiological data for the user collected by a wearable computing device.

14. The method of claim 13, wherein the method further comprises generating the inferred exercise pattern using the plurality of exercise events as input into a classifier that identifies a pattern formed by exercise events with common characteristics.

15. The method of claim 8, wherein the method further comprises disambiguating location data provided by a smart phone that corresponds to an exercise venue with physiological data collected contemporaneously with the smart phone being located at the exercise venue to determine that an exercise event did not occur.

16. One or more computer-storage media comprising computer-implemented instructions that when executed by a computer processor cause a computer to perform a method of inferring an exercise pattern comprising:
receiving location signal data indicating a location of a mobile computing device associated with a user;
receiving physiological signal data describing a physiological state of the user;
using an exercise event inference engine to identify an exercise event by analyzing the location signal data and the physiological signal data together, the exercise event comprising an event context;
storing a record of the exercise event in an exercise event data store that comprises a plurality of exercise events records;
using an exercise pattern inference engine to identify the exercise pattern by finding exercise events having contextual features in common, the exercise pattern associated with a periodic context and a behavioral context; and
storing a description of the exercise pattern in an exercise pattern data store.

17. The media of claim 16, further comprising:
determining that the behavioral context is not satisfied at a time when the periodic context indicates a probable future exercise event is to occur; and
suggesting an alternative exercise event to the user.

18. The media of claim 17, wherein the behavioral context is a calendar with no entries scheduled within a threshold time of the time when the periodic context indicates a probable future exercise event is to occur.

19. The media of claim 17, wherein the user is associated with multiple exercise patterns and the alternative exercise event is from a different pattern associated with the user.

20. The media of claim 16, wherein the behavioral context is being located within a home city.
